(19) United States
(12) Patent Application Publication
(10) Pub. No.: US 2006/0171909 A1
(43) Pub. Date: Aug. 3, 2006

(54) Cosmetic compositions comprising colorants with low free dye

(75) Inventors: Christopher Todd Morrissey, Mason, OH (US); Karen Kay Kalla, Cincinnati, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH (US)

(21) Appl. No.: 11/050,210

(22) Filed: Feb. 3, 2005

(51) Int. Cl. A61K 8/19

(52) U.S. Cl. 424/63

(57) ABSTRACT

Cosmetic compositions suitable for changing the appearance of keratinaceous substances, such as mammalian skin, hair, and nails. The compositions comprise an encapsulated colorant having a high encapsulation efficiency, which mitigates colorant staining of the keratinaceous substance, and a particle size that yields the desired color.

### FIELD OF THE INVENTION

The present invention is directed to cosmetic compositions suitable for changing the appearance of keratinaceous substances, such as mammalian skin, hair, and nails. The compositions comprise an encapsulated colorant. The encapsulate has a high encapsulation efficiency, which mitigates colorant staining of the keratinaceous substance, and a particle size that yields the desired color.

### BACKGROUND OF THE INVENTION

Perception of color is determined by the observation of light reflecting across visible wavelengths, specifically within the range of about 400-700 nm. To measure the perceived color of an object, a spectrophotometric curve (also termed “spectral curve”) may be plotted wherein the percentage of reflectance is measured versus the wavelength of light.

After a period of time, the exterior appearance of human skin becomes discolored due to various factors such as environmental exposure, aging, and basic human physiology. In an attempt to revitalize and duplicate the youthful yet natural and original properties of skin, various cosmetic compositions have been developed, including, for example, foundations and concealers. Typically, the cosmetic compositions are applied to the skin to cover imperfections and/or simulate healthy or natural-looking skin. It is therefore desirable to provide cosmetic compositions which exhibit the natural qualities exhibited by skin and particularly which exhibit the color tone of skin, thereby appearing less noticeable and more natural when applied to skin.

Despite this desired effect, however, many current cosmetic compositions do not substantially match the color and tone of skin. As a result, the human eye is visually able to detect the presence of such cosmetic compositions once they are applied, whereby an unnatural skin look is perceived. Accordingly, there is a desire for providing cosmetic compositions that better match the color of skin and have a natural skin-like appearance. Additionally, it is desirable for these compositions to have enhanced color and to provide sufficient coverage once applied to skin while remaining virtually undetectable to the naked eye, i.e., look like youthful skin.

Colorants are used to provide permanent, semi-permanent, and temporary color to personal care products for the hair, skin, and nails. Often, the desired colorant is a dye, which can stain or color the desired substrate. In many applications, staining is undesirable, thus limiting the use of dyes. Therefore, dyes are typically converted to lakes which render the dye mostly insoluble. However, in the presence of water or another suitable solvent, some of the dye is leached from the lake, leaving staining potential. It is difficult to achieve high dye retention without covalent dye attachment, which is undesired in cosmetic applications. Obtaining the desired colorant characteristics of low free dye and a small particle size are also challenging. Therefore, colorants with staining potential are limited in their use level. This results in products with several potential drawbacks, including not being able to produce a bright, saturated color or having a low hiding power, or constrast ratio. Thus, there still exists a need to reduce or mitigate the temporary staining such that consumers can wipe the product off their skin, hair, and nails and leave no noticeable stain prior to washing, while still acheving the desired beauty benefit.

### SUMMARY OF THE INVENTION

The present invention provides a cosmetic composition comprising a colorant. The colorant is preferably an encapsulate with a high encapsulation efficiency, which mitigates colorant staining when used on a keratinaceous substance. Furthermore, the high encapsulation efficiency can increase the color stability of the product.

In one embodiment, the composition comprises colorants that are particularly well suited to providing natural skin tone by better matching the spectral profile of the skin. The colorants have low extractable dye content and do not stain skin. In addition, the colorants are well dispersed.

In a particular embodiment, the cosmetic composition comprises an encapsulate, wherein said encapsulate comprises a colorant, and further wherein said encapsulate has an encapsulation efficiency of greater than about 40%. In a particular embodiment, the encapsulate is a polymeric encapsulate. In another embodiment, the colorant comprises a mixture of two or more colorants.

As used herein, the term “encapsulate” means at least one encapsulate. Thus, the term “encapsulate” is broad enough to include not only one encapsulate, but also to a mixture of more than one encapsulate.

As used herein, “cosmetic composition” means any color cosmetic, hair, nail, or skin care product. “Cosmetic compositions” include, but are not limited to, products that leave color on the face, including foundation, blacks and browns (i.e., mascara), concealers, eye liners, brow colors, eye shadows, blushers, lip sticks, lip balms, face powders, solid emulsion compact, and so forth. The term “foundation” refers to liquid, cream, mousse, pancake, compact, concealer or like product created or reintroduced by cosmetic companies to even out the overall coloring of the skin.

As used herein, both “spectrophotometric curve” and “spectral curve” refer generally to a plotted curve displaying an ordinate value of relative reflectance versus an abscissa value of wavelength of light, typically within the visual range of about 400-700 nm.

All percentages, parts and ratios are based upon the total weight of the compositions of the present invention, unless otherwise specified. All such weights as they pertain to listed ingredients are based on the active level and, therefore do not include solvents or by-products that may be included in commercially available materials, unless otherwise specified. The term “weight percent” may be denoted as “wt. %” herein.

All measurements made are at 25° C., unless otherwise designated.

### DETAILED DESCRIPTION OF THE INVENTION

In one aspect, the present invention provides a cosmetic composition that better mimics the spectral curve of skin. In one embodiment, the composition comprises:
(a) iron oxide; and
(b) an encapsulate, wherein said encapsulate comprises:
   (1) a core shell architecture;
   (2) an aqueous core; and
   (3) a dye encapsulated in said aqueous core, and
wherein said encapsulate has an encapsulation efficiency of greater than about 40%; and
wherein said encapsulate comprises a dye.
   In a particular embodiment, the average size of the encapsulate is less than about 5 microns, preferably less than about 3 microns. In one embodiment, the encapsulate has an encapsulation efficiency of greater than about 60%, preferably greater than about 90%.

In another aspect, the present invention provides a cosmetic composition for changing the appearance of mammalian keratinaceous substances. In one embodiment, the composition comprises an encapsulate, wherein said encapsulate comprises:
(a) a core shell architecture;
(b) an aqueous core; and
(c) a dye encapsulated in said aqueous core, and

wherein said encapsulate has an encapsulation efficiency of greater than about 40%, preferably greater than about 60%, and more preferably greater than about 90%. In a particular embodiment, the encapsulate has a particle size average of less than about 5 microns, preferably less than about 3 microns. In a particular embodiment, the composition comprises at least about 0.2% encapsulate.

The compositions of the present invention can comprise a water soluble dye, and in one embodiment preferably a water soluble dye that comprises an acidic group. In a particular embodiment, the water soluble dye can be selected from the group consisting of F&DC Yellow 5, D&C Yellow 10, D&C Red 33, D&C Green 5, D&C Yellow 8, D&C Red 28, FD&C Blue #1, Carmine, and mixtures thereof.

Furthermore, the compositions can comprise an encapsulate that comprises a covalently bound dispersant group.

In another aspect, the present invention provides compositions that can have a spectrophotometric curve with a first derivative in which: a) a maximum peak in the region of from about 430 nm to about 520 nm occurs at a wavelength not greater than about 480 nm; b) a maximum peak in the region of from about 420 nm to about 650 nm occurs at a wavelength of from about 570 nm to about 630 nm; and c) a minimum valley in the region of from about 520 nm to about 580 nm has Δ%R/Δλ of less than or equal to about 0.03, wherein R is reflectance and λ is wavelength, and wherein the cosmetic composition comprises a mixture of at least two colorants, wherein a first derivative of the spectrophotometric curve of each of the individual colorants does not exhibit (a), (b) and (c).

In a particular embodiment, the invention provides a cosmetic composition for changing the appearance of mammalian keratinaceous substances, wherein said composition comprises an encapsulate having a particle size average of less than about 5 microns, preferably less than about 3 microns, and wherein said encapsulate has an encapsulation efficiency of greater than about 40%; and further wherein said composition has a spectrophotometric curve, wherein the composition has a spectrophotometric curve with a first derivative in which: a) a maximum peak in the region of from about 430 nm to about 520 nm occurs at a wavelength not greater than about 480 nm; b) a maximum peak in the region of from about 420 nm to about 650 nm occurs at a wavelength of from about 570 nm to about 630 nm; and c) a minimum valley in the region of from about 520 nm to about 580 nm has Δ%R/Δλ of less than or equal to about 0.03, wherein R is reflectance and λ is wavelength, and wherein the cosmetic composition comprises a mixture of at least two colorants, wherein a first derivative of the spectrophotometric curve of each of the individual colorants does not exhibit (a), (b) and (c).

The invention also provides a method for reducing skin staining by using an encapsulated colorant with high encapsulation efficiency, comprising applying a composition of the present invention to a keratinaceous substance, such as the skin, hair, or nails.

Non limiting examples in the art can be modified by one skilled in the art to include the present invention and improve the utility of the art; examples of such art that can utilize the present invention herein can include: EP0833601B1, EP0862412B1, EP1204396A1, EP1204398A1, US6340466B1, US6406683B1, US6475500B2, US6482398B1, US6524598B2, US6696049B2, US20020018760A1, US20030049212A1, US20030108498A1, US20040086473A1, US20040086474A1, WO0203933A2, WO2003080005A1, WO2003105787A2, WO2003105790A1, EP1389085A2, US4996044A, US5505937A, US6682748B1, US5234682A, US6042815A, US6080390A, US6103250A, US6120780A, US6458390B1, US6726900B2, WO2002094182A2, and WO2002096352A2

A. Colorant

The composition of the present invention comprises a colorant. In general, colorants are those substances that provide color to a personal care product. The purpose of the colorant is to deliver the desirable shade or color that the user is seeking and often to even out skin tone by covering or hiding tonal imperfections. Such colorants should be physically and chemically compatible with the essential components described herein, or should not otherwise unduly impair product stability, aesthetics or performance.

The concentration of the colorants generally ranges from about 0.001% to about 50%, preferably from about 0.005% to about 20%, more preferably from about 0.01% to about 10%, by weight of the composition. Suitable colorants for use in the composition herein include pigments, dyes, free dyes, and mixtures thereof.

Pigments are defined as colorants that are insoluble in the medium in which they are being used. Thus, pigments do not substantially dissolve or are insoluble in product or usage. Often, pigments are slightly soluble in the product. This soluble portion of the pigment is referred to as free dye. Pigments include, but are not limited to, lakes and encapsulated colorants.

Dyes are colorants that are substantially soluble in the medium in which they are being used. The use of dyes is often intended to provide permanent, semi-permanent or durable color for the hair, skin, or nails. However, if sufficient concentration of dye is present, skin staining can occur. This staining is usually not durable and can be washed off or easily removed, and the stain will usually fade over time. However, a high concentration of a dye can yield a more durable stain.

Some of the dyes which can be used herein include, but are not limited to, D&C Yellow No. 7, D&C Red No. 36, FD&C Red No. 4, D&C Orange No. 4, D&C Red No. 6, D&C Red No. 34, FD&C Yellow No. 6, D&C Red No. 33, FD&C Yellow No. 5, D&C Brown No. 1, D&C Red No. 17, FD&C Green No. 3, D&C Blue No. 4, D&C Yellow No. 8, D&C Orange No. 5, D&C Red No. 22, D&C Red No. 21, D&C Red No. 28, D&C Orange No. 11, D&C Yellow No. 10, D&C Violet No. 2, Ext. D&C Violet No. 2, D&C Green No. 6, D&C Green No. 5, D&C Red No. 30, D&C Green No. 8, D&C Red No. 7, FD&C Blue No. 1, D&C Red No. 27, D&C Orange No. 10, D&C Red No. 31, FD&C Red No. 40, D&C Yellow No. 11, CI 10020, CI 16185, CI 16255, CI 45430, CI 73015, CI 74160, carmine, and mixtures thereof.

Water soluble dyes, identified by one skilled in the art, are dyes that are substantially soluble in aqueous solutions. For the purposes of this invention, acid dyes are dyes that contain acidic groups, especially sulphonic acid groups. Preferred examples of water soluble acid dyes include, but are not limited to, D&C Red 33, FD&C Yellow No. 5, D&C Green No. 5, D&C Yellow No. 8, D&C Yellow No. 10, and FD&C Blue No. 1.

Colorants that do not have free dye may also be used in conjunction with the pigments above. Some of these useful herein include, but are not limited to, aluminum powder, ultramarines, bismuth oxychloride, chromium oxide green, chromium hydroxide green, iron oxides, ferric ferrocyanide, manganese violet, titanium dioxide, zinc oxide, mica, bronze powder, copper powder, aluminum stearate, calcium stearate, magnesium stearate, zinc stearate, capsanthin/capsorubin, bentonite, barium sulfate, calcium carbonate, calcium sulfate, carbon black, magnesium carbonate, colored silica, and mixtures thereof. Other suitable colorants and pigments may be found in the International Cosmetic Ingredient Dictionary and Handbook, Seventh Edition.

B. Encapsulation

Encapsulation generally involves sequestering dyes or pigments in a solid substrate. These substrates include polymers and other solids such as silica. A variety of encapsulation methods including core-shell are known in the field. Any suitable encapsulation method can be useful herein, and non-limiting methods are described in the following patents and applications: U.S. Pat. No. 3,196,079, DE 1913569, JP 73020302, JP 49075738, JP 50049169, U.S. Pat. No. 4,880,617, JP 59076009, JP 59128322, JP 61047410, JP 61083109, JP 61166827, EP 212870, EP 225799, EP 232001, JP 62209011, U.S. 4,756,906, EP 238225, JP 62254833, U.S. Pat. No.4,665,107, JP 62234541, JP 63060914, WO 03/015910, JP 63171678, JP 63179972, JP 63196505, JP 63202671, EP 306331, JP 1175920, JP 2164439, JP 3034910, U.S. Pat. No. 5,234,711, WO 9106277, JP 3221137, EP 445342, JP 3293028, EP 522916, JP 5339518, WO 9305753, JP 4001118, JP 10059818, WO 9818867, WO 9850002, U.S. Pat No. 6,060,084, EP 922449, JP 11197494, WO 9943426, and WO 9962974.

The use of colorants, especially encapsulated colorants, with free dye levels of greater than about 40%, when used at levels of greater than about 0.1% solids level or higher, can lead to noticeable staining. Higher use levels of the colorants used in the present invention, however, are possible with less visicble staining than compositions in the past. This allows a larger array of colors to be produced than was previously possible.

Colorants with small particle size are also desired for the production of a wide array of colors and increased color vibrancy. In general, smaller particle size colorants hve higher amounts of free dyes. This is especially true of encapsulated colorants. This is a further limitation of current materials. The preferred materials the present invention have a high ecapsulation efficiency and particule size of less than about 5 microns, more preferably below about 3 microns.

A preferred encapsulant has a capsule core comprising at least one colorant, and a capsule shell, wherein the shell comprises reaction products of: (a) at least one di-, oligo- and/or polyisocyanate, and (b) at least one polyfunctional amine slected from the group consisting of polyvinylamines, polyethylenimines and polyoxyalkylenamines having a number-average molecular weight of from about 600 to about 380,000 g/mol, and (c) optionally, one or more alkyldiamines having 2 to 10 carbon atoms. This preferred encapsulant is available from BASF Aktiengelellschaft, and is described in BASF's WO patent applications entitled “Microcapsule Dispersions” and “Low Viscosity Microcapsule Dispersions,” filed on the same date as the present application.

This preferred encapsulant comprises a capsule shell and a capsule core. The capsule core comprises at least one colorant, preferably a water-soluble colorant, in solid form and/or, as a result of preparation, in the form of a solution in a hydrophilic solvent. Preferred capsule cores comprise solutions of the colorant.

By reactants in reference to this preferred encapsulant are meant at least one polyfunctional amine having an average molecular weight of from about 600 to about 380,000 g/mol and the alkyldiamine having 2 to 10 carbon atoms, to be used if desired, as compounds which react with di-, oligo- and/or polyisocyanate groups.

The basic principle of microencapsulation is based on what is called interfacial addition polymerization or interfacial polyaddition. With interfacial polyaddition, in a first process step, the materials for encapsulation and the ractants, as they are known, are dissolved in a hydrophilic solvent, after which a hydrophobic solvent is added and the system is processed to an emulsion. The continuous phase of the emulsion normally includes surface-active substance, preventin coalescene of the droplets. Within this emulsion the hydrophilic solvent is the discontinuous, disperse phase and the hydrophobic solvent is the continuous phase. Where the hydrophilic solvent is water, the ter water-in-oil emulsion is also illustrative. The emulsified droplets possess a size that corresponds approximately to the size of the subsequent microcapsules. To form the capsule wall in a second process step of the emulsion is mixed with the isocyanate capable of wall forming. The reactants are capable of reacting with the isocyanate in solution in the continuous phase, at the interface between the discontinuous and continuous phases, to form the polymeric capsule wall.

The third step of the process, which may optionally be carried out, comprises what is called the aftertreatment of the freshly prepared capsule dispersion. In this step, under temperature and residence time control and, if desired, using further auxiliaries, the reaction between isocyanate and reactant is carried out to completion.

In another preferred embodiment of encapsulant, also disclosed in the aforementioned BASF applications, the encapsulates comprise a capsule core, comprising at least one colorant, and a capsule shell, obtainable by:

a) interfacial polyaddition of at least one di, oligo, and/or polyisocyanate with at least one reagent carrying at least one iscyanate-reactive group and

b) subsequent aftertreatment of the primary product capsules with at least one compound selected from the group consisting of amines, alcohols and amino alcohols having a molecular weight of at least 150 g/mol and

c) if appropriate, subsequent aftertreatment with at least one further aftertreatment reagent

The capsules comprise a capsule shell and a capsule core. The capsule core comprises at least one water-soluble organic substance in solid form and/or, as a result of its preparation, in the form of solution in a suitable solvent. The capsule core preferably comprises a water soluble dye in the form of a hydrophilic solution.

A reactant can be a compound containing at least one isocyanate-reactive group. Preferred reactants are those whose isocyanate-reactive groups are OH, NH and/or NH2 groups which are able to react with isocyanate groups. Preferred reactants among these include the primary amines.

Particularly preferred reactants are the polyfunctional amines such as, for example, the polyvinylamines, the polyoxyalkyleneamines and/or the polyethylenimines. Particularly preferred reactants of these are those having a number-average molecular weight of from about 600 to about 380,000 g/mol. These reactants may also be used in the form of mixtures, particularly in the form of mixtures with at least one alkyldiamine having 2 to 10 carbon atoms.

The basic principle of this encapsulatioin is based on what is called interfacial addition polymerization or interfacial polyaddition. With interfacial polyaddition, in a first process step, the materials for encapsulation and the reactant, as it is known, are dissolved, for example, in a hydrophilic solvent after which a hydrophobic solvent is added and the system is process to an emulsion. The continuous phase of the emulsion normally includes surface-active substances, preventing coalescence of the droplets. Within this emulsion the hydrophilic solvent is the discontinous, dispersed phase and the hydrophobic solvent is the continuous phase. Where the hydrophilic solvent is water, the term water-in-oil emulsion is also illustrative. The emulsified droplets possess a size that corresponds to the approximate size of the subsequent capsules. To form the capsule wall in a second process step, the emulsion is mixed with the di-, oligo-, and/or polyisocyante capable of wall forming. The reactant is capable of reacting with the isocyanate in solution in the continuous phase, at the interface between the discontinuous and continuous phases, to form the polymeric capsule walls.

The next step of this process comprises what is called the aftertreatment of the freshly prepared capsule dispersion. In this step, under temperature and residence time control, the reaction of the isocyanate functions of the di-, oligo- and/or polyisocyanate(s) introduced that have not reacted with the OH, NH, and/or NH2 functions of the reactant(s) employed is completed. For the aftertreatment according to step b) at least one compound is added selected from the group consisting of amines, alcohols and amino alcohols having a molecular weight of at least 150 g/mol. The free isocyanate groups still present are reacted with the selected aftertreatment reagent, ie.e an amine, alcohol or amino alcohol or mixture thereof. Preference is given to using aftertreatment reagents which have a number average molecular weight of from about 200 to about 70,000 g/mol, mor preferably from about 200 to about 20,000 g/mol, in particular from about 300 to about 10,000 g/mol and most preferred from about 400 to about 5000 g/mol. Mention may be made by way of example of aftertreatment reagents which can be used in accordance with the invention of the following compounds: aminated fatty alcohols such as stearylamine, oleylamine, arachidylamine, and laurylamine, for example, and aminated C30-C50 alcohols such as cetyl amine, nonatrien-1-amine, isotridencylamine and behenylamine, for example. Also, suitable aftertreatments examples include, polyoxyalkylenemonomines such as XTJ-505 (M-600), XTJ-506 (M-1000), XTJ-507 (M2005) and Jeffamine M-2070 (all Huntsman). Furthermore, polyisobutyleneamines such as Kerocom PIBA 03 (˜1000 g/mol from BASF) and those specified in EP-A 0244616.

Free dye can be measured by extraction and measurement of the free dye. The free dye of the encapsulated colorants of the present invention is measured according to the following method, in terms of the encapsulation efficiency (“ee”). The method may be modified or adjusted as necessary for different colorants/dyes.

0.2 g of a uniformly mixed sample of the encapsulated colorant is weighed into a 50 ml centrifuge tube (polyethylene). 10 ml of an extraction solution (1:1 mixture of fully deionized water and 2-propanol) are added to the sample. The solution is mixed thoroughly and then centrifuged for 20 minutes. Thereafter the supernatant solution is transferred to a glass beaker. The wash extraction process is repeated until the supernatant liquid is colorless. The collected wash solutions are made up to 100 ml with the extraction solution. One portion of the collected solution is filtered through a 0.2 nm filter and the amount of encapsulated colorant is determined by UV spectroscopy using a UV-VIS spectrometer from HP (HP 8453). The encapsulation efficiency is calculated by the following formula:
Encapsulation efficiency=((A−B)/A)*100
where A is the total amount of colorant for encapsulation present in the analyzed sample and B is the product of the UV-spectroscopically determined concentration and the volume of the analyzed sample.
And:
Free dye=Encapsulation Efficiency−100
C. Optional Ingredients

The compositions of the present invention can comprise any suitable optional ingredients, such as those described in U.S. patent application Ser. No. 10/840833, filed May 7, 2004; and in U.S. application Ser. No. 10/174247, filed Jun. 18, 2002 and published as U.S. Application Publication No. 2003/0003064A1 on Jan. 2, 2003. Optional ingredients can include, but are not limited to, aqueous carriers, desquamation actives (such as those disclosed in U.S. Pat. No. 5,681,852), anti-acne actives, antiperspirant actives, anti-wrinkle/anti-atrophy actives, anti-oxidants/radical scavengers, chelators, flavonoids (such as those disclosed in U.S. Pat. Nos. 5,686,082 and 5,686,367), anti-inflammatory agents, anti-cellulite agents, topical anesthetics, tanning actives, skin lightening agents (such as those described in PCT Publication No. 95/34280, PCT Application No. 95/07432, and PCT Publication No. 95/23780), skin soothing actives, skin healing actives, antimicrobial actives, antifungal actives, sunscreen actives (such as those disclosed by Sagarin, et al., at Chapter VIII, pages 189 et seq., of Cosmetics Science and Technology (1972)), particulate materials (such as those disclosed in U.S. Pat. No. 5,997,887), conditioning agents (such as those described in U.S. Pat. No. 4,976,953), thickening agents (such as those described in U.S. Pat. No. 5,087,445, U.S. Pat. No. 4,509,949, U.S. Pat. No. 2,798,053, and in CTFA International Cosmetic Ingredient Dictionary, Fourth Edition, 1991, pp. 12 and 80; U.S. Pat. No. 5,100,660, U.S. Pat. No. 4,849,484, U.S. Pat. No. 4,835,206, U.S. Pat. No. 4,628,078, U.S. Pat. No. 4,599,379, EP 228,868, U.S. Pat. Nos. 5,654,362 and 5,880,210; Warth, Chemistry and Technology of Waxes, Part 2, Reinhold Publishing, 1956)), additional powdered ingredients (such as those described in C.T.F.A. Cosmetic Ingredient Handbook, First Edition, Washington D.C. (1988), and in U.S. Pat. No. 5,505,937), materials for enhancing wear or transfer resistance (such as those disclosed by E. S. Barabas in the Encyclopedia of Polymer Science and Engineering, 2 Ed., Vol. 17, pp. 198-257; PCT Publication Nos. WO96/33689 and WO97/17058, and U.S. Pat. No. 5,505,937, PCT publication No. WO98/18431, and U.S. Pat. No. 5,800), emulsifiers (such as those described by Wilkinson and Moore, Harry's Cosmeticology, 7th Ed. 1982, p. 738; Schick and Fowkes, Surfactant Science Series, Vol. 2, Solvent Properties of Surfactant Solutions, p 607; C.T.F.A. Cosmetic Ingredient Handbook, 3rd Ed., Cosmetic and Fragrance Assn., Inc., Washington D.C. (1982) pp. 587-592; Remington's Pharmaceutical Sciences, 15th Ed. 1975, pp. 335-337; McCutcheon's Volume 1, Emulsifiers & Detergents, 1994, North American Edition, pp. 236-239, U.S. Pat. No. 4,268,499, and U.S. Pat. No. 5,143,722), and co-solubilizers (such as those described by U.S. Pat. No. 4,268,499 and U.S. Pat. No. 5,143,722).

D. Spectrophotometric Curve

As used herein, both “spectrophotometric curve” and “spectral curve” refer generally to a plotted curve displaying an ordinate value of relative reflectance versus an abscissa value of wavelength of light, typically within the visual range of about 400-700 nm. As used herein, the “first derivative” of the spectrophotometric curve or spectral curve refers generally to a plotted curve displaying the ordinate values of Δ%R/Δλ versus an abscissa value of wavelength of light, typically within the visual range of about 400-700 nm, wherein R is reflectance and λ is wavelength. It is generally known that various perceived colors can be compared, particularly by measuring and plotting the reflectance of light across visible wavelengths to produce a spectral curve. Once the spectral curves of the perceived colors are produced, it is then possible to compare the measured curves for any distinguishing color characteristics that are exhibited by the individual colors. Spectral curves can be measured by any number of methods known to those skilled in the art. This spectral curve is typically measured at every 10 nm between 400-700 nm by a number of instruments. Particularly noted are the instruments from Data Color International that yield spectral curve in a reflectance mode. Spectrophotometric curves are described in US 2003/0003065 A1. Preferably, the composition of the present invention better mimics the spectral curve of skin. Most preferably, the composition of the present invention mimics the spectral curve of skin.

One method of determining that the invention better mimics the spectral curve is to calculate the difference in area between a spectral curve of the skin target and the product of interest. The product is said to better mimic the skin if the area between the product and target product is smaller than the area between the spectral curves of a control product and skin at a specific wavelength range. Appropriate choice of the ratios of the colorants of the invention and iron oxides results in a foundation with improved match to a target skin curve.

Compositions of the present invention are said to mimic the spectral curve of skin when the compositions have a spectrophotometric curve with a first derivative in which: (a) a maximum peak in the region of from about 430 nm to about 520 nm occurs at a wavelength not greater than about 480 nm; (b) a maximum peak in the region of from about 420 nm to about 650 nm occurs at a wavelength of from about 570 nm to about 630 nm; and (c) a minimum valley in the region of from about 520 nm to about 580 nm has Δ%R/Δλ of less than or equal to about 0.03, wherein R is reflectance and λ is wavelength, and wherein the cosmetic composition comprises a mixture of at least two colorants, wherein a first derivative of the spectrophotometric curve of each of the individual colorants does not exhibit (a), (b) and (c).

E. Delivery Means

As mentioned herein relative to the phrase “cosmetic compositions”, the cosmetic compositions of the present invention may be adapted for delivery to a defined surface via a delivery mechanism. For example, suitable delivery mechanisms include, but are not limited to, colloidal systems, delivery substrates (i.e., applicator pads, sponges, open cell foams, brushes), packages, spraying devices and printing devices. More specifically, delivery devices such as electrostatic spraying devices, air-spraying devices, ink jet print heads, lithographic devices or stamp pads are suitable for use herein. Non-limiting examples include EP0443741B1, U.S. Pat. No. 5,903,465, U.S. Pat. No. 6,510,366, U.S. Pat. No. 6,516,245, and U.S. Pat. No. 6,615,881.

The cosmetic compositions of the invention are adapted for application to a substrate to provide an applied cosmetic composition having a spectrophotometric curve whose first derivative exhibits desirable characteristics. In such embodiments, the cosmetic composition adapted for application is not required to itself have a spectrophotometric curve whose first derivative exhibits the desired maximum and minimum characteristics. Rather, it is only required that the composition is adapted to provide, for example, via a delivery mechanism, an applied cosmetic composition whose first derivative of the spectrophotometric curve exhibits them. Thus, in this instance the delivery mechanism alters the cosmetic composition in such a manner prior to application to the target substrate that the desired characteristics are achieved upon delivery.

More particularly, this embodiment includes cosmetic compositions adapted for application to a substrate to provide an applied cosmetic composition having a spectrophotometric curve, wherein the applied cosmetic composition comprises at least two colorants, and wherein the composition has a spectrophotometric curve with a first derivative in which: (a) a maximum peak in the region of from about 430 nm to about 520 nm occurs at a wavelength not greater than about 480 nm, (b) a maximum peak in the region of from about 420 nm to about 650 nm occurs at a wavelength of from about 570 nm to about 630 nm, and (c) a minimum valley in the region of from about 520 nm to about 580 nm has a Δ%R/Δλ of less than or equal to about 0.03; wherein R is reflectance and λ is wavelength, and wherein a first derivative of a spectrophotometric curve of each of the individual colorants does not exhibit (a), (b) and (c). In such instances, it is preferred that Δ%R/Δλ is less than or equal to about 0.015, and most preferably, less than or equal to about 0.

In a specific embodiment, the cosmetic compositions of the invention have a spectrophotometric curve whose first derivative exhibits characteristic maximum and minimum that are substantially similar to those exhibited by the first derivative of the spectral curve of skin. For example, in an exemplary embodiment of the present invention, the cosmetic compositions are provided with skin tone color and have a spectrophotometric curve with a first derivative in which: (a) a maximum peak in the region of from about 430 nm to about 520 nm occurs at a wavelength not greater than about 480 nm, (b) a maximum peak within the region of from about 420 nm to about 650 nm that occurs at a wavelength of from about 570 nm to about 630 nm, and (c) a minimum valley in the region of from about 520 nm to about 580 nm has a Δ%R/Δλ of less than or equal to about 0.03; wherein R is reflectance and λ is wavelength, and wherein the cosmetic composition comprises a mixture of at least two colorants, wherein a first derivative of a spectrophotometric curve of each of the individual colorants does not exhibit (a), (b) and (c). Preferably, Δ%R/Δλ is less than or equal to about 0.015, and most preferably, less than or equal to about 0.

F. Methods for Providing Cosmetic Compositions and Associated Methods of Use

The present invention also relates to various methods for providing cosmetic compositions having a desired spectral curve exhibiting maximum and minimum first derivative characteristics. In one embodiment, a cosmetic composition with a skin tone color is provided by a method that comprises adding at least two colorants to the cosmetic composition. In a specific embodiment, the step of adding at least two colorants may comprise delivering to the cosmetic composition at least two discrete color domains as described above. In another specific embodiment, the step of adding at least two colorants comprises physically admixing at least two different colorants with the cosmetic composition. Colorants suitable for such physical mixing are those described above.

In another embodiment, the invention is directed to methods for providing a colorant mixture to spectrophotometrically match a cosmetic composition to a target substrate like skin. Methods according to this embodiment comprise: i) determining a mixture of colorants adapted to provide a cosmetic composition having a spectrophotometric curve, wherein the composition has a spectrophotometric curve with a first derivative in which: (a) a maximum peak in the region of from about 430 nm to about 520 nm occurs at a wavelength not greater than about 480 nm; (b) a maximum peak in the region of from about 420 nm to about 650 nm occurs at a wavelength of from about 570 nm to about 630 nm; and (c) a minimum valley in the region of from about 520 nm to about 580 nm has a Δ%R/Δλ of less than or equal to about 0.03, wherein R is reflectance and λ is wavelength; and ii) admixing the colorants to form a mixture; wherein a first derivative of a spectrophotometric curve of each of the individual colorants does not exhibit (a), (b) and (c). In a specific embodiment, the method may further comprise the step of adding the mixture to the cosmetic composition. As used herein, “target substrate” according to these methods may include, but is not limited to, mammalian skin, particularly human skin.

In a further embodiment, the invention includes methods for providing discrete color domains for spectrophotometrically matching a cosmetic composition to a target substrate. These methods comprise the steps of: i) determining a combination of colorants which form discrete color domains adapted to provide a cosmetic composition having a spectrophotometric curve, wherein the composition has a spectrophotometric curve with a first derivative in which: (a) a maximum peak in the region of from about 430 nm to about 520 nm occurs at a wavelength not greater than about 480 nm; (b) a maximum peak in the region of from about 420 nm to about 650 nm occurs at a wavelength of from about 570 nm to about 630 nm; and (c) a minimum valley in the region of from about 520 nm to about 580 nm has a Δ%R/Δλ of less than or equal to about 0.03; wherein R is reflectance and λ is wavelength; and ii) depositing the colorants into at least two discrete color domains; wherein a first derivative of a spectrophotometric curve of each of the individual colorants does not exhibit (a), (b) and (c). In a specific embodiment, methods may further comprise the step of adding the color domains to the cosmetic composition. In each of the aforementioned methods as well as those that follow, it is preferred that Δ%R/Δλ is less than or equal to about 0.015, and most preferred that it is less than or equal to about 0.

Applicants have found that the compositions of the present invention are useful in a variety of applications directed to enhancement of mammalian skin, hair, and nails. The methods of use for the compositions disclosed and claimed herein include, but are not limited to: 1) methods of increasing the substantivity of a cosmetic to skin; 2) methods of moisturizing skin; 3) methods of improving the natural appearance of skin; 4) methods of applying a color cosmetic to skin; 5) methods of preventing, retarding, and/or treating wrinkles; 6) methods of providing UV protection to skin; 7) methods of preventing, retarding, and/or controlling the appearance of oil; 8) methods of modifying the feel and texture of skin; 9) methods of providing even skin tone; 10) methods of preventing, retarding, and/or treating the appearance of spider vessels and varicose veins; 11) methods of masking the appearance of vellus hair on skin; 12) methods of concealing blemishes and/or imperfections in human skin, including acne, age spots, freckles, moles, scars, under eye circles, birth marks, post-inflammatory hyperpigmentation; 13) methods of enhancing or modifying skin color such as lightening, darkening, making more pink, making more yellow, making less dull, making less ashy, making less orange, making more radiant; 14) methods of artificial tanning; 15) methods of concealing vitiligo; 16) methods of concealing damage incurred to the skin as a result of trauma, e.g., cosmetic surgery, burns, stretching of skin, etc.; and 17) methods of concealing wrinkles, fine lines, pores, uneven skin surfaces, etc.; and 18) methods of masking the appearance of cellulite. Each of the methods discussed herein involve topical application of the claimed compositions to skin.

### EXAMPLES

The following are non-limiting examples of the compositions of the present invention. The examples are given solely for the purpose of illustration and are not to be construed as limitations of the present invention, as many variations thereof are possible without departing from the spirit and scope of the invention, which would be recognized by one of ordinary skill in the art. In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the present invention as described herein.

As used herein, the abbreviation “ee” means “encapsulation efficiency.”

### Examples 1 & 2

| Materials | Example 1 | Example 2 |
|---|---|---|
| Titanium Dioxide | 8.25% | 8.25% |
| Preservative | 0.5% | 0.5% |
| Cyclomethicone & | 45% | 45% |
| Dimethicone crosspolymer | | |
| Cyclopentasiloxane & | 2.4% | 2.4% |
| Dimethicone copolyol | | |
| Dimethicone copolyol | 2.5% | 2.5% |
| crosspolymer | | |
| Encapsulated colorant | 14% | 14% |
| premix | (low encapsulation | (high encapsulation |
| | efficiency) | efficiency) |
| Water | 8% | 8% |
| Glycerin | 8% | 8% |
| Cyclopentasiloxane | q.s. | q.s. |

Encapsulated Colorant Premixes

| Low Encapsulation Efficiency | | High Encapsulation Efficiency | |
|---|---|---|---|
| Capric/Capryllic | 60% | Capric/Capryllic | 60% |
| Triglyceride | | Triglyceride | |
| D&C Green 5 encapsulate | 3.2 | D&C Green 5 encapsulate | 4% |
| 62% ee | | 92% ee | |
| D&C Red 33 encapsulate | .05 | D&C Red 33 encapsulate | 0.2% |
| 43% ee | | 95% ee | |
| FD&C Yellow 5 | 6 | FD&C Yellow 5 | 8.4% |
| encapsulate 22% ee | | encapsulate 90% ee | |
| D&C Yellow 10 16% ee | 5 | D&C Yellow 10 92% ee | 10.4% |
| Carmine encapsulate 91% | 25.75 | Carmine encapsulate 90% | 17% |
| ee | | ee | |

Particle size average < 5 μm

| | |
|---|---|
| 1 | Phase A - Mix, TiO2, silicone copolyol crosspolymer and optionally |
| | silicone phase preservatives with the cyclopentasiloxane and mill |
| | until TiO2 is well dispersed |
| 2 | Phase B - Separately, mix water, glycerin, and preservatives until |
| | uniform, heating if necessary |
| 3 | Add phase B to Phase A, mill until uniform |
| 4 | Add Dimethicone crosspolymer and mix until uniform |
| 5 | Add Encapsulate Colorant Premix and mix until uniform. |
| 6 | Pass sample through 3 roll-mill - one pass |

The formula of Example 1 results in more noticeable staining than that of Example 2.

### Example 3

### Foundation with Iron Oxides and Encapsulated Colorants

| Materials | Amount |
|---|---|
| Titanium Dioxide | 3.99% |
| Preservative | 0.68% |
| Cyclomethicone & Dimethicone crosspolymer | 40% |
| Cyclopentasiloxane & Dimethicone copolyol | 2.17% |
| Dimethicone copolyol crosspolymer | 2.1% |
| Encapsulated colorant premix | 22.4% |
| Yellow Iron Oxide 55% Slurry - Kobo | 0.61% |
| Black Iron Oxide 65% Slurry - Kobo | 0.50% |
| Red Iron Oxide 70% Slurry - Kobo | 0.21% |
| Water | 8.8% |
| Glycerin | 8.8% |
| Cyclopentasiloxane | q.s. |

Encapsulated Colorant Premix
Material with Low Encapsulation Efficiency

| Capric/Capryllic Triglyceride | q.s. |
|---|---|
| D&C Green 5 encapsulate 92% ee | 2.0 |
| D&C Red 33 encapsulate 95% ee | 3.96 |
| FD&C Yellow 5 encapsulate 90% ee | 8.74 |
| D&C Yellow 10 92% ee | 11.1 |
| Carmine encapsulate 90% ee | 10.84 |

Particle size average < 5 μm

| | |
|---|---|
| 1 | Phase A - Mix, TiO2, silicone copolyol crosspolymer and optionally |
| | silicone phase preservatives with the cyclopentasiloxane and mill |
| | until TiO2 is well dispersed |
| 2 | Phase B - Mix water, glycerin, and preservatives until uniform, |
| | heating if necessary |
| 3 | Add phase B to Phase A, mill until uniform |
| 4 | Add Dimethicone crosspolymer and mix until uniform |
| 5 | Add Encapsulate Colorant Premix and mix until uniform. |
| 6 | Pass sample through 3 roll-mill - one pass |

### Example 4

### Foundation with Iron Oxides—Comparative Example

| Materials | Amount |
|---|---|
| Titanium Dioxide | 2.00% |
| Preservative | 0.75% |
| Cyclomethicone & Dimethicone crosspolymer | 45% |
| Cyclopentasiloxane & Dimethicone copolyol | 3.9% |
| Dimethicone copolyol crosspolymer | 0.5% |
| Yellow Iron Oxide 55% Slurry - Kobo | 2.23% |
| Black Iron Oxide 65% Slurry - Kobo | 0.68% |
| Red Iron Oxide 70% Slurry - Kobo | 0.75% |
| Water | 10% |
| Glycerin | 10% |
| Cyclopentasiloxane | q.s. |

| | |
|---|---|
| 1 | Phase A Combine pigments, TiO2, and silicone phase ingredients. |
| | Mill until well dispersed |
| 2 | Mix water, glycerin, and preservatives until uniform, heating if |
| | necessary |
| 3 | Add phase B to Phase A, mill until uniform |
| 4 | Add Dimethicone crosspolymer and mix until uniform |
| 5 | Pass sample through 3 roll-mill - one pass |

In comparison to the formulation of Example 4, that of Example 3 better mimics the spectral curve of human skin, especially in the 7550 nm range.

### Example 5

### Lipstick

| Materials | Percentage |
|---|---|
| Isopropyl Isostearate | 15 |
| Octyl Hydroxystearate | 8.5 |
| Acetylated Lanolin | 6.33 |
| Ozokerite was | 5 |
| Candelilla wax | 3 |
| Paraffin Wax | 2.5 |
| Carnauba Wax | 2 |
| Cetyl Alcohol | 2 |
| Cetyl Lactate | 2 |
| Ascorbyl Palmitate | 0.5 |
| Propylparaben | 0.1 |
| TiO₂ | 1.92% |
| Lipstick colorant premix | 26.35% |
| Castor Oil | q.s. |

Lipstick colorant premix

| Capric/Capryllic Triglyceride | q.s. |
|---|---|
| D&C Red 33 encapsulate ee > 60% | 16.8% |
| FD&C Yellow 5 encapsulate ee > 60% | 6.0% |
| D&C Yellow 10 encapsulate ee > 60% | 7.2% |
| Carmine encapsulate ee > 60% | 10% |

| | |
|---|---|
| 1 | Mix ingredients in under low shear with heat (˜70-80° C.) until |
| | uniform |
| 2 | Remover air under reduced pressure |
| 3 | Pour molten mixture into mold |
| 4 | Cool |
| 5 | Remove from mold and place in appropriate package |

### Example 6

### Liquid Lip Color

| Ingredient | Percentage |
|---|---|
| Organo silioxane resin (MQ resin 0.7:1 M:Q ratio - GE) | 20.84 |
| Dimethicone gum (100,000-1,000,000 cSt - GE) | 14.03 |
| Hectorite clay | 3.09 |
| Propylene Carbonate | 0.93 |
| Isododecane | q.s. |
| TiO₂ | 2.04 |
| Liquid Lip Colorant premix | 25 |
| | 100 |

Procedure:
1) MQ resin and Dimethicone gum are dissolved into the appropriate amount of isododecane solvent
2) Isododecane, hectorite clay and propylene carbonate are milled into a paste
3) The paste, resin and gum mixture are combined and milled.
4) TiO2 is added to the above mixture and milled until uniform.

5) Colorant premix is added to the above TiO2 monochromatic mixture and milled

Liquid Lip Colorant Premix

| Capric/Capryllic Triglyceride | q.s. |
|---|---|
| D&C Red 33 encapsulate ee > 60% | 16.8% |
| FD&C Yellow 5 encapsulate ee > 60% | 6.0% |
| D&C Yellow 10 encapsulate ee > 60% | 7.2% |
| Carmine encapsulate ee > 60% | 10% |

### Example 7

### Tinted Moisturizer

| Ingredient | Wt % |
|---|---|
| Cyclomethicone & Dimethicone crosspolymer | 45.00 |
| Cyclopentasiloxane & Dimethicone copolyol | 5.00 |
| TiO₂ | 2 |
| Tinted Moisturizer Colorant Premix | 3.5 |
| Cyclomethicone | q.s. |
| Preservatives | 0.7 |
| Water | 15.00 |
| Glycerin | 10.00 |

Tinted Moisturizer Colorant Premix

| Capric/Capryllic Triglyceride | q.s. |
|---|---|
| D&C Green 5 encapsulate 92% ee | 4% |
| D&C Red 33 encapsulate 95% ee | 0.2% |
| FD&C Yellow 5 encapsulate 90% ee | 8.6% |
| D&C Yellow 10 92% ee | 11% |
| Carmine encapsulate 90% ee | 16% |

| | |
|---|---|
| 1 | Phase A - Mix, TiO2, silicone copolyol crosspolymer and optionally |
| | silicone phase preservatives with the cyclopentasiloxane and mill |
| | until TiO2 is well dispersed |
| 2 | Phase B - Separately, mix water, glycerin, and preservatives until |
| | uniform, heating if necessary |
| 3 | Add phase B to Phase A, mill until uniform |
| 4 | Add Dimethicone crosspolymer and mix until uniform |
| 5 | Add Tinted Moisturizer Colorant Premix and mix until uniform. |
| 6 | Pass sample through 3 roll-mill - one pass |

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

All documents cited herein are incorporated by reference in their entirety. The citation of any document is not to be construed as an admission that it is prior art with respect to the present invention.

What is claimed is:

1. A cosmetic composition that better mimics the spectral curve of skin, wherein said composition comprises:
(a) iron oxide; and
(b) an encapsulate, wherein said encapsulate comprises:
(1) a core shell architecture;
(2) an aqueous core; and
(3) a dye encapsulated in said aqueous core, and
wherein said encapsulate has an encapsulation efficiency of greater than about 40%; and
wherein said encapsulate comprises a dye.

2. The composition of claim 1, where the average particle size of said encapsulate less than about 5 microns.

3. The composition of claim 2, where the average particle size of said encapsulate less than about 3 microns.

4. The composition of claim 2, wherein said encapsulate has an encapsulation efficiency of greater than about 60%.

5. The composition of claim 2, wherein said encapsulate has an encapsulation efficiency of greater than about 90%.

6. A cosmetic composition for changing the appearance of mammnalian keratinaceous substances, wherein said composition comprises at least about 0.2% encapsulate, wherein said encapsulate comprises:
(a) a core shell architecture;
(b) an aqueous core; and
(c) a dye encapsulated in said aqueous core, and
wherein said encapsulate has an encapsulation efficiency of greater than about 40%.

7. The composition of claim 6, having an encapsulation efficiency of greater than about 60%.

8. The composition of claim 7, having an encapsulation efficiency of greater than about 90%.

9. The composition of claim 6, wherein said composition comprises an encapsulate having a particle size average of less than about 5 microns.

10. The composition of claim 6, wherein said encapsulate has a particle size average of less than about 3 microns.

11. The composition of claim 1, wherein said water soluble dye comprises an acidic group.

12. The composition of claim 6, wherein said water soluble dye comprises an acidic group.

13. The composition of claim 1, wherein said water soluble dye is selected from the group consisting of F&DC Yellow 5, D&C Yellow 10, D&C Red 33, D&C Green 5, D&C Yellow 8, D&C Red 28, FD&C Blue #1, Carmine, and mixtures thereof.

14. The composition of claim 6, wherein said water soluble dye is selected from the group consisting of F&DC Yellow 5, D&C Yellow 10,D&C Red 33, D&C Green 5, D&C Yellow 8, D&C Red 28, FD&C Blue #1, Carmine, and mixtures thereof.

15. The composition of claim 1, wherein said encapsulate comprises a covalently bound dispersant group.

16. The composition of claim 6, wherein said encapsulate comprises a covalently bound dispersant group.

17. The composition of claim 6, wherein said composition has a spectrophotometric curve with a first derivative in which: a) a maximum peak in the region of from about 430 nm to about 520 nm occurs at a wavelength not greater than about 480 nm; b) a maximum peak in the region of from about 420 nm to about 650 nm occurs at a wavelength of from about 570 nm to about 630 nm; and c) a minimum valley in the region of from about 520 nm to about 580 nm has Δ%R/Δλ of less than or equal to about 0.03, wherein R is reflectance and λ is wavelength, and wherein the cosmetic composition comprises a mixture of at least two colorants, wherein a first derivative of the spectrophotometric curve of each of the individual colorants does not exhibit (a), (b) and (c).

18. A cosmetic composition for changing the appearance of mammalian keratinaceous substances, wherein said composition comprises an encapsulate having a particle size average of less than about 5 microns and wherein said encapsulate has an encapsulation efficiency of greater than about 40%; and further wherein said composition has a spectrophotometric curve with a first derivative in which: a) a maximum peak in the region of from about 430 nm to about 520 nm occurs at a wavelength not greater than about 480 nm; b) a maximum peak in the region of from about 420 nm to about 650 nm occurs at a wavelength of from about 570 nm to about 630 nm; and c) a minimum valley in the region of from about 520 nm to about 580 nm has Δ%R/Δλ of less than or equal to about 0.03, wherein R is reflectance and λ is wavelength, and wherein the cosmetic composition comprises a mixture of at least two colorants, wherein a first derivative of the spectrophotometric curve of each of the individual colorants does not exhibit (a), (b) and (c).

19. The composition of claim 18, wherein said encapsulate has a particle size average of less than about 3 microns.

20. A method for reducing skin staining by using an encapsulated colorant with high encapsulation efficiency, comprising applying the composition of claim 18 to a keratinaceous substance.
